(19) United States
(12) Patent Application Publication
(10) Pub. No.: US 2025/0114234 A1
(43) Pub. Date: Apr. 10, 2025

(54) FLUID COLLECTION SYSTEMS INCLUDING AN ON-DEMAND PUMP, AND RELATED METHODS

(71) Applicant: PUREWICK CORPORATION, Covington, GA (US)

(72) Inventors: Edward Espinosa, Ocala, FL (US); Aaron Van Gorkom, Lynnwood, WA (US); Brendan Bolos, Oceanside, CA (US); Rebecca Weiss, Atlanta, GA (US); Clayton Lewis Compton, Conyers, GA (US); Michael Zohil, Loganville, GA (US); Marian Gravel, Newburgh, NY (US); Paul Riyad, Palm Beach Gardens, FL (US); Samuel Hernandez, Covington, GA (US); James Saunders, Lowell, MA (US); Brian Moyer, Covington, GA (US); Maciej Pietrusinski, Covington, GA (US); Michael Guarino, Covington, GA (US); Jameson Moore, Covington, GA (US); Marya Schnedeker, Covington, GA (US); Jason Lasser, Covington, GA (US)

(21) Appl. No.: 18/836,204

(22) PCT Filed: Feb. 9, 2023
(86) PCT No.: PCT/US2023/012696

§ 371 Date: Aug. 6, 2024

Related U.S. Application Data
(60) Provisional application No. 63/308,190, filed on Feb. 9, 2022.

(51) Int. Cl. A61F 5/455; A61M 1/00

(52) U.S. Cl. CPC A61F 5/455; A61M 1/80

(57) ABSTRACT

Example fluid collection system, and related methods of use are described. The fluid collection system includes a fluid collection device having a chamber and configured to collect fluid from a user in the chamber. The fluid collection system includes a first conduit in fluid communication with the chamber of the fluid collection device. The fluid collection system includes a handheld pump unit connected or connectable to the first conduit and including a pump configured to create a negative pressure to draw fluid from the chamber of the fluid collection device through the first conduit. The fluid collection system includes a fluid storage container having an interior region in fluid communication with the first conduit and configured to collect fluid drawn from the chamber of the fluid collection device through the first conduit.

### CROSS-REFERENCE TO RELATED APPLICATIONS

This application claims priority to U.S. Provisional Patent Application No. 63/308,190 filed on Feb. 9, 2022, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine and other fluids.

### SUMMARY

Embodiments disclosed herein are fluid collection systems and related methods of use. In an embodiment, a fluid collection system includes a fluid collection device having a chamber and configured to collect fluid from a user in the chamber. The fluid collection system includes a first conduit in fluid communication with the chamber of the fluid collection device. The fluid collection system includes a handheld pump unit connected or connectable to the first conduit and including a pump configured to create a negative pressure to draw fluid from the chamber of the fluid collection device through the first conduit. The fluid collection system includes a fluid storage container having an interior region in fluid communication with the first conduit and configured to collect fluid drawn from the chamber of the fluid collection device through the first conduit.

In an embodiment, a fluid collection system includes a fluid collection device, a first conduit, a handheld pump unit, and a fluid storage bag. The fluid collection device has a chamber and is configured to collect fluid from a user in the chamber. The first conduit is in fluid communication with the chamber of the fluid collection device. The handheld pump unit is connected or connectable to the first conduit and includes a pump configured to create a negative pressure to draw fluid from the chamber of the fluid collection device through the first conduit. The fluid storage bag has an interior region in fluid communication with the first conduit and is configured to collect fluid drawn from the chamber of the fluid collection device through the first conduit. The handheld pump includes a depth of about 5 cm to about 12 cm, a width of about 6.5 cm to about 13 cm, and a height of about 4 cm to about 14 cm.

In an embodiment, a fluid collection system includes a fluid collection device, a conduit, a handheld pump unit, and a rigid fluid storage container. The fluid collection device has a chamber and configured to collect fluid from a user in the chamber. The conduit is in fluid communication with the chamber of the fluid collection device. The handheld pump unit is connected or connectable to the conduit and includes a pump configured to create a negative pressure to draw fluid from the chamber of the fluid collection device through the conduit. The rigid fluid storage container is secured or securable to the pump unit and has an interior region in fluid communication with the conduit and is configured to collect fluid drawn from the chamber of the fluid collection device through the first conduit. When the pump unit is secured to the rigid fluid storage container, the pump unit and the rigid fluid storage container together include a width of about 10.5 cm to about 12.5 cm, a depth of about 6.5 cm to about 8.5 cm, and height of about 17.5 cm to about 19.5 cm.

In an embodiment, a method of collecting fluid utilizing any of the fluid collection systems described herein is disclosed.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a system for fluid collection, according to an embodiment.

FIG. 2 is a front isometric view of a fluid collection device, according to an embodiment.

FIG. 3A is an isometric view of a fluid collection system, according to an embodiment.

FIG. 3B is an isometric view of a pump unit of the fluid collection system of FIG. 3A.

FIG. 3C is an isometric view of a plug adapter of the fluid collection system of FIG. 3A.

FIG. 4A is an isometric view of a fluid collection system, according to an embodiment.

FIG. 4B is an isometric view of a pump unit of the fluid collection system of FIG. 4A.

FIGS. 5A-5B are isometric views of a fluid collection system, according to an embodiment.

FIG. 5C is an isometric view of an assembly including a pump unit and rigid fluid storage container of the fluid collection system of FIGS. 5A-5B.

### DETAILED DESCRIPTION

Described herein are systems and methods for collecting fluid from an individual. Fluid collection systems may include a pump, a fluid storage container, a fluid collection device, and one or more conduits providing fluid communication between the fluid collection device, the fluid storage container, and/or the pump. At least one, some, or all embodiments of fluid collection systems and related methods described herein include a pump configured for use with individuals having toileting challenges related to mobility limitations. In at least one, some, or all embodiments, the pump connects to a capture/catheter device (e.g., fluid collection device) and moves the urine away from the body to at least one of a toilet or a fluid collection canister/bag (e.g. fluid storage container).

Women and men who have full control of their bladder (e.g., know when they “need to go”) often have functional incontinence and/or limited or lack of mobility. These women and men may endure challenges with urine and other fluid management throughout the day and night, for which conventional fluid collection systems do not meet their emotional and physical needs. Conventional fluid collection systems, for example, may lead to an increased risk of skin damage, obligates the users to hold an urge to urinate, and/or obligates users to deliberately dehydrate themselves. At least one, some, or all embodiments of pumps of fluid collection systems described herein result in the technical effect of an increased level of independence and improved quality-of-life for the users of the fluid collection systems. For example, at least one, some, or all embodiments of pumps described herein may provide an on-demand urine management solution for women or men with limited mobility and functional incontinence to afford the women or men greater independence, reduce fall/injury risk, and/or reduce the need for a caregiver/nurse during the toileting process. At least one, some, or all embodiments of pumps described herein may be configured as a reusable pump that is selectively used intermittently (e.g., less than 5 minutes) and includes an on/off switch.

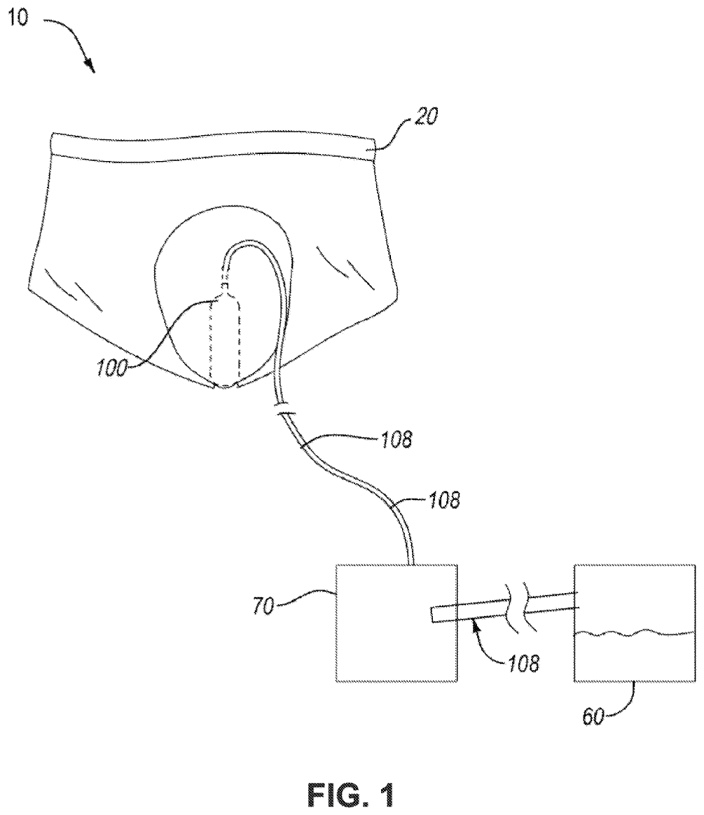

FIG. 1 is a block diagram of a system 10 for fluid collection, according to an embodiment. The system 20 includes a fluid collection device 100, a fluid storage container 60, and a vacuum source 70 (e.g., pump). The fluid collection device 100, the fluid storage container 60, and the vacuum source 70 may be in fluid communication with each other via one or more conduits 108. For example, fluid collection device 100 may be operably coupled to (e.g., in fluid communication with) one or more of the fluid storage container 60 and/or the vacuum source 70 via the conduits 108. A garment 20 (e.g., undergarment) may be worn by a user (not shown) and may interface at least a portion of the fluid collection device 100. Embodiments of garments are disclosed in PCT Patent Application No. PCT/US21/44699, filed on Aug. 5, 2021, the disclosures of which are incorporated herein, in its entirety, by this reference.

Fluid (e.g., urine, vaginal discharge, penile discharge, reproductive fluids, blood, sweat, or other bodily fluids.) collected in the fluid collection device 100 may be removed from the fluid collection device 100 via the conduit 108 which protrudes into an interior region of the fluid collection device 100. For example, a first open end of the conduit 108 may extend into the fluid collection device 100 to a reservoir therein. The second open end of the conduit 108 may extend into the fluid storage container 60 or the vacuum source 70. The suction force may be introduced into the interior region of the fluid collection device 100 via the first open end of the conduit 108 responsive to a suction (e.g., vacuum) force applied at the second end of the conduit 108. The suction force may be applied to the second open end of the conduit 108 by the vacuum source 70 either directly or indirectly. In the system 10 of FIG. 1, the suction force is applied to the fluid collection device 100 by the vacuum source 70 via the conduit 108. The vacuum source 70 may then direct (e.g. force) fluid pulled from the fluid collection device 100 to the fluid storage container 60.

The suction force may be applied indirectly via the fluid storage container 60. For example, the second open end of the conduit 108 may be disposed within the fluid storage container 60 and an additional conduit 108 may extend from the fluid storage container 60 to the vacuum source 70. Accordingly, the vacuum source 70 may apply suction to the fluid collection device 100 via the fluid storage container 60. In some examples, the suction force may be applied directly via the vacuum source 70. For example, the first open end of the conduit 108 may be disposed in the fluid collection device 100 and the second open end of the conduit 108 may be disposed within the vacuum source 70. An additional conduit 108 may extend from the vacuum source 70 to a point outside of the fluid collection device 100, such as to the fluid storage container 60. In such examples, the vacuum source 70 may be disposed between the fluid collection device 100 and the fluid storage container 60.

In examples, the fluid storage container 60 may include a bag (e.g., drainage bag), a bottle or cup (e.g., collection jar), tubing, or any other container for storing bodily fluids such as urine. In examples, the conduit 108 may extend from the fluid collection device 100 and attach to the fluid storage container 60 at a first point therein. An additional conduit 108 may attach to the fluid storage container 60 at a second point thereon and may extend and attach to the vacuum source 70. For example, the fluid storage container 60 may include a container in fluid communication with a first conduit section that is also in fluid communication with the fluid collection device 100. The container may be in fluid communication with a second section of the conduit 108 that is also in fluid communication with the vacuum source. In such examples, the vacuum source 70 may provide a vacuum/suction through the container to the fluid collection device 100 to provide suction in the chamber of the fluid collection device. Accordingly, a vacuum (e.g., suction) may be drawn through fluid collection device 100 via the fluid storage container 60. As the fluid is drained from the chamber, the fluid may travel through the first section of conduit to the fluid storage container where it may be retained. Fluid, such as urine, may be drained from the fluid collection device 100 using the vacuum source 70.

The vacuum source 70 may include one or more of a manual vacuum pump, and electric vacuum pump, a diaphragm pump, a centrifugal pump, a displacement pump, a magnetically driven pump, a peristaltic pump, or any pump configured to produce a vacuum (e.g., negative pressure). The vacuum source 70 may include any of the pumps described in greater detail herein. The vacuum source 70 may provide a vacuum or suction to remove fluid from the fluid collection device 100. In examples, the vacuum source 70 may be powered by one or more of a power cord (e.g., connected to a power socket), one or more batteries, or even manual power (e.g., a hand operated vacuum pump). The vacuum sources 70 disclosed herein may include one or more of a switch, a button, a plug, a remote, or any other device suitable to activate the vacuum source 70.

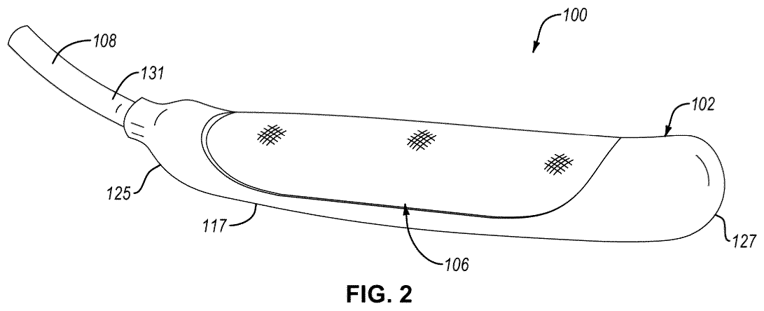

FIG. 2 is an isometric view of the fluid collection device 100, according to an embodiment. The fluid collection device 100 is an example of a female fluid collection device 100 that is configured to receive fluids from a female. The urine collection device 100 may be configured to be positioned at least proximate to a urethra of a user. While the urine collection device 100 shown in FIG. 2 includes a female urine collection device, the urine collection device 100 may instead include a male urine collection device. PCT International Application No. PCT/US2019/029616, for example, describes various embodiments of both male and female fluid collection devices, the disclosure of which are incorporated in their entirety by this reference. Moreover, the urine collection device 100 may be interchangeable in the urine collection system 100 between different types, varieties, and sizes of male or female urine collection devices. Generally, the urine collection device 100 may include a surface sized to be positioned proximate or adjacent to the urethra and configured to wick urine or other fluids away from the user. Urine or other fluids may be wicked from the surface to a reservoir in the urine collection device 100.

The fluid impermeable barrier 102 may include a front region 115 positioned to orient towards the user during use and a back or rear region 117 positioned to orient away from the user during use. The fluid impermeable barrier 102 at least partially defines a chamber (e.g., interior region,) and includes an inward border or edge 129 on the front regions 115 defining an opening 106. The opening 106 is formed in and extends longitudinally through the fluid impermeable barrier 102, thereby enabling fluids to enter the chamber 104 from outside of the fluid collection device 100. The opening 106 may be configured to be positioned adjacent to the opening of a female urethra.

The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. For example, in some embodiments, the front region 115 is generally arched or rounded and the back region 117 is generally planar. According to various embodiments, the fluid impermeable barrier 102 may include any of a number of different shapes and configurations, such as the shapes and configurations of the fluid impermeable barriers disclosed in U.S. Patent Application No. 63/171,165 filed on Apr. 6, 2021, U.S. Patent Application No. 63/228,258 filed on Aug. 2, 2021, U.S. Patent Application No. 63/228,252 filed on Aug. 2, 2021, U.S. Patent Application No. 63/228,244 filed on Aug. 2, 2021, U.S. patent application Ser. No. 17/394,055 filed Aug. 4, 2021, or PCT Application No. PCT/US20/40860, the disclosures of each of which are incorporated herein by reference in their entirety.

The fluid collection device 100 may be positioned at least proximate to the opening of the female urethra or over the urethra of a male having a buried penis, and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir of void space formed within the chamber 104 and/or towards the inlet 110 of the conduit 108. For example, the fluid permeable body 120 may include a porous or fibrous material, such as spun nylon fibers or hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface of the fluid permeable body 120 interfaces with an inner surface of the fluid impermeable barrier 106. In some embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable support, with both the fluid permeable membrane and the fluid permeable support being disposed in the chamber. The fluid permeable membrane may cover or extend across at least a portion (e.g., all) of the opening 106. The fluid permeable membrane may be configured to wick any fluid away from the opening 106, thereby preventing the fluid from escaping the chamber. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating. Such “wicking” or other physical properties may not include absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption is desired, the term “substantially no absorption” may allow for nominal amounts of absorption of fluid into the wicking material (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 120. In an embodiment, the fluid permeable body 120 may include at least one absorbent or adsorbent material.

The fluid permeable membrane may also wick the fluid generally towards an interior of the chamber. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous nylon structure (e.g., spun nylon fibers) and a fluid permeable membrane including gauze about or over the porous nylon structure of the fluid permeable support.

In use, the fluid permeable body 120 of the fluid collection device is positioned adjacent to a urethra of the user. The fluid permeable body 120 is disposed within a chamber of the fluid impermeable barrier 102 of the fluid collection device 100 and is exposed to the urethra of the user through the opening 106 in the fluid collection device 100. The fluid collection device 100 may be secured to the user with any of a number of securing devices. Fluids received in the chamber of the fluid collection device 100 from the urethra may be removed through the conduit 108.

The fluid collection device 100 also includes the conduit 108 that is at least partially disposed in the chamber 104. The conduit 108 (e.g., a tube) includes an inlet at a second end region 127 of the fluid impermeable barrier 102 and an outlet at a first end region 125 of the fluid impermeable barrier 102 positioned downstream from the inlet. The conduit 108 provides fluid communication between an interior region of the chamber and a fluid storage container (not shown) or a portable vacuum source (not shown). For example, the conduit 108 may directly or indirectly fluidly couple the interior region of the chamber and/or the reservoir with the fluid storage container or the portable vacuum source.

The fluid impermeable barrier 102 may store fluids in the reservoir or sump therein. According to some embodiments, the reservoir is an unoccupied portion of the chamber 104 and is void of other material. In some embodiments, the reservoir is defined at least partially by the fluid permeable body 120 and the fluid impermeable barrier 102. For example, in an embodiment, the reservoir may be located at the portion of the chamber that is closest to the inlet (e.g., the second end region). However, the reservoir may be located at different locations in the chamber. For example, the reservoir may be located at the end of the chamber 104 that is closest to the outlet. In these and other embodiments, the conduit 108 may extend through the first end region 125 of the fluid impermeable barrier 102 and to the reservoir without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber that is closest to the inlet (e.g., second end region) and a second reservoir that is located at the portion of the of the chamber that is closest to the outlet (e.g., first end region). In another example, the fluid permeable body 120 is spaced from at least a portion of the conduit 108 and the reservoir may be the space between the fluid permeable body 120 and the conduit 108. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber. For example, the fluid permeable body 120 may be configured to form a space that accommodates the conduit 108, such as the bore. In another example, the fluid impermeable barrier 102 may define an aperture sized to receive the conduit 108 (e.g., at least one tube). The at least one conduit 108 may be disposed in the chamber via the aperture. The aperture may be configured to form an at least substantially fluid tight seal against the conduit 108 or the at least one tube thereby substantially preventing the fluids from escaping the chamber.

In some embodiments, the conduit 108 may extend through the fluid permeable body 120 and at least partially into the reservoir. In some embodiments, the conduit 108 may extend through the fluid permeable body 120 and terminate at or before the second body end of the fluid permeable body 120 such that the conduit 108 does not extend into the reservoir (or the reservoir is absent of the conduit 108). For example, an end of the conduit 108 may be generally flush or coplanar with the second body end of the fluid permeable body 120. In other embodiments, the end of the conduit 108 may be recessed from the second body end of the fluid permeable body 120. The end of the conduit 108 also may be selectively moveable between partially extending into the reservoir 122 and recessed from or flush with the second body end of the fluid permeable body.

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet and the outlet are configured to provide fluid communication (e.g., directly or indirectly) between the portable vacuum source (not shown) and the chamber (e.g., the reservoir). For example, the inlet and the outlet of the conduit 108 may be configured to directly or indirectly fluidly couple the portable vacuum source or pump to the reservoir. In an embodiment, the inlet and/or the outlet may form a male connector. In another example, the inlet and/or the outlet may form a female connector. In an embodiment, the inlet and/or the outlet may include ribs that are configured to facilitate secure couplings. In an embodiment, the inlet and/or the outlet may form a tapered shape. In an embodiment, the inlet and/or the outlet may include a rigid or flexible material.

Locating the inlet at or near a gravimetrically low point of the chamber enables the conduit to receive more of the fluids than if inlet was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids. As the vacuum source applies a vacuum/suction in the conduit 108, the fluid(s) in the chamber (e.g., such as in the reservoir positioned at the first end region 125, the second end region 127, or other intermediary positions within the chamber) may be drawn into the inlet and out of the fluid collection device 100 via the conduit 108.

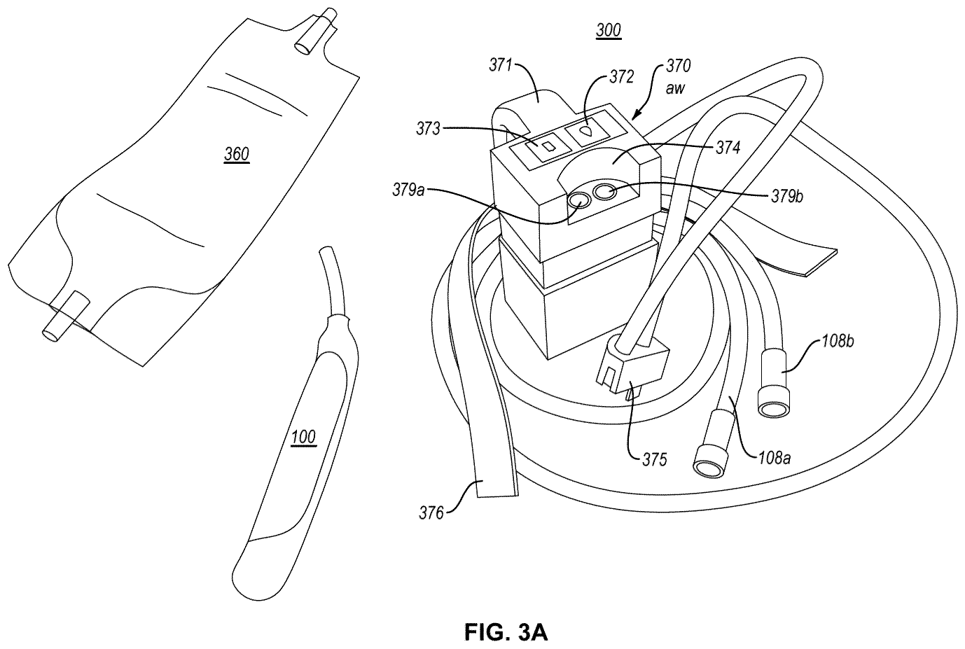

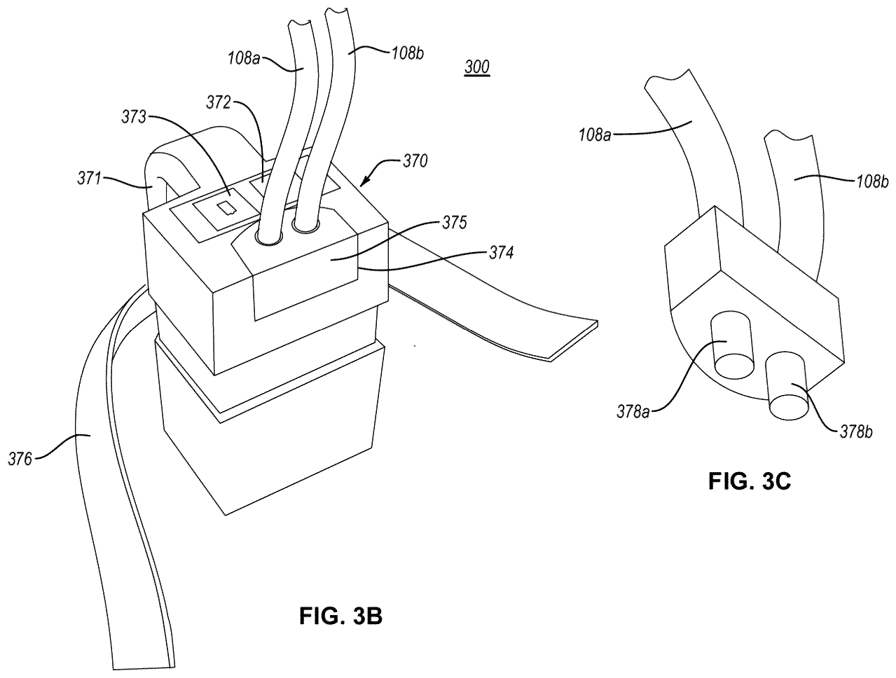

Turning now to FIG. 3A, a disassembled fluid collection system 300 including a fluid collection device 100, an inlet conduit 108a, and outlet conduit 108b, a fluid storage container 360 (e.g., bag), and a pump unit 370 according to an embodiment are provided. FIG. 3A, for example, shows an embodiment of a disassembled fluid collection system 300 including a pump unit 370, an inlet conduit 108a disconnected from the pump 370, an outlet conduit 108b disconnected from the pump unit 370, the fluid collection device 100 disconnected from the inlet conduit 108a, and a fluid storage bag 360 (e.g. fluid storage container) disconnected from the outlet conduit 108b. In some embodiments, the pump unit 370 and the fluid storage bag 360 are separate components from one another than can be selectively and fluidly connected with one another via one or more conduits 108.

The pump unit 370 may house any of the pumps described herein. The pump unit 370 may include a rechargeable and/or selectively removable battery. The pump unit 370 is sized, dimensioned, and weight to be handheld by the user. In some embodiments, the pump unit 370 includes a handle 371 protruding from one or more sides or surfaces of the pump unit 370. The handle 371 may be sized, dimensioned, and otherwise configured to allow the pump unit 370 to hook onto an armrest or a cup holder (e.g., a cup holder built into furniture). In some embodiments, the pump unit 370 may be sized and dimensioned to fit at least partially within a cup holder to all the cup holder to support the pump.

Turning to FIG. 3B, the pump unit 370 may include a height of about 13.3 cm, a width of about 7.6 cm, and a depth of about 5.7 cm, according to an embodiment. In some embodiments, the pump unit 370 may include a height of about 10.2 cm to about 15.2 cm, about 10.2 cm to about 12.7 cm, about 12.7 cm to about 15.2 cm, about 10.2 cm to about 11.4 cm, about 11.4 cm to about 12.7 cm, about 12.7 cm to about 14 cm, about 14 cm to about 15.2 cm, less than about 15.2 cm, less than about 14 cm, less than about 12.7 cm, or less than about 11.4 cm. In some embodiments, the pump unit 370 may include a width of about 5.1 cm to about 10.2 cm, about 5.1 cm to about 7.6 cm, about 6.4 cm to about 8.9 cm, about 7.6 cm to about 10.2 cm, about 5.7 cm to about 7 cm, about 7 cm to about 8.3 cm, about 8.3 cm to about 9.5 cm, less than about 10.2 cm, less than about 8.9 cm, less than about 7.6 cm, or less than about 6.4 cm. In some embodiments, the pump unit 370 may include a depth of about 2.5 cm to about 7.6 cm, about 2.5 cm to about 5.1 cm, about 5.1 cm to about 7.6 cm, about 3.8 cm to about 5.1 cm, about 5.1 cm to about 6.4 cm, about 6.4 cm to about 7.6 cm, less than about 7.6 cm, less than about 6.4 cm, or less than about 5.1 cm.

Returning to FIG. 3A, the pump unit 370 may include a user interface, shown at the top of the pump unit in FIG. 3A. The user interface may include an on/off button 372 that is configured to selectively activate (e.g., turn on) the pump in the pump unit 370 and selectively deactivate (e.g., turn off) the pump in the pump unit 370. The user interface also may include a battery display 373 that is configured to indicate at least one of when the battery needs to be charged, when the battery is fully charged, or a status of the battery of the pump unit 370. In some embodiments, the pump unit 370 includes an LED light as the battery indicator. The LED light may display certain colors for levels of battery power (e.g., green indicates a charged battery, yellow indicates low battery, and red indicates an empty or depleted battery needing to be charged).

The pump unit 370 may be fluidly coupled to the fluid collection device 100 with a first or inlet conduit 108a and fluidly coupled to the fluid storage bag 360 with a second or outlet conduit 108b. The inlet conduit 108a may include an adapter configured to secure to a conduit extending from the fluid collection device 100 effective to provide fluid communication between the chamber of the fluid collection device 100 and the inlet conduit 108a. The outlet conduit 108bmay include an adapter configured to secure to the fluid storage bag 360 effective to provide fluid communication between an interior region of the fluid storage bag 360 and the outlet conduit 108b. In operation, the pump unit 370 may be downstream from the fluid collection device 100, and the fluid storage bag 360 may be downstream from the pump unit 370 (e.g., the pump unit 370 is positioned between the fluid storage bag 360 and the fluid collection device 100). The pump unit 370, then, may create a negative pressure in the inlet conduit 108a to pull fluid from the fluid collection device 100, then direct fluid through the outlet conduit 108b to the fluid storage bag 360.

In some embodiments, the conduits 108a, 108b may be configured with adapters that prevent or inhibit dripping or leaking for fluids. For example, turning to FIG. 3C, the pump unit may include an adapter plug 375 (shown removed from the ports 379a, 379b in the recess or receiver 374 of FIG. 3A). The inlet conduit 108a and the outlet conduit 108b may extend from the adapter plug 375. The adapter plug 375 is configured to detachably secure to (e.g. plug into) the pump unit 370 effective provide fluid communication between the ports 379a, 379b and the inlet conduit 108a and the outlet conduit 108b. For example, the adapter plug 375 may include a first prong 378a configured to slide into a first port 379a effective provide fluid communication between the pump in the pump unit 370 and the inlet conduit 108b. The adapter plug 375 also may include a second prong 378b configured to slide into a second port 379b effective provide fluid communication between the pump in the pump unit 370 and the outlet conduit 108b.

FIG. 3C illustrates the plug adapter 375 removed from the pump unit 370, and FIG. 3B illustrates the plug adapter 375 secured to the pump unit 370 in the recess 374. With the plug adapter 375 plugged into the recess 374 of the pump unit 370 that includes the ports 379a, 379b, upon activation, the pump unit 370 may create a negative pressure in the inlet conduit 108a and direct fluid pulled from the inlet conduit 108a into the outlet conduit 108b. In some embodiments, the inlet conduit 108a and the outlet conduit 108b (e.g., tubing) may be clear or translucent. The fluid storage bag 360 may include a clear disposable bag.

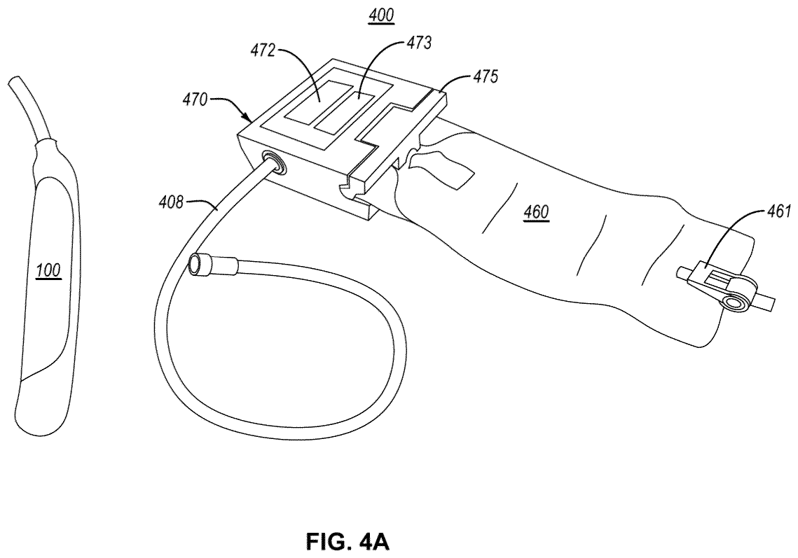

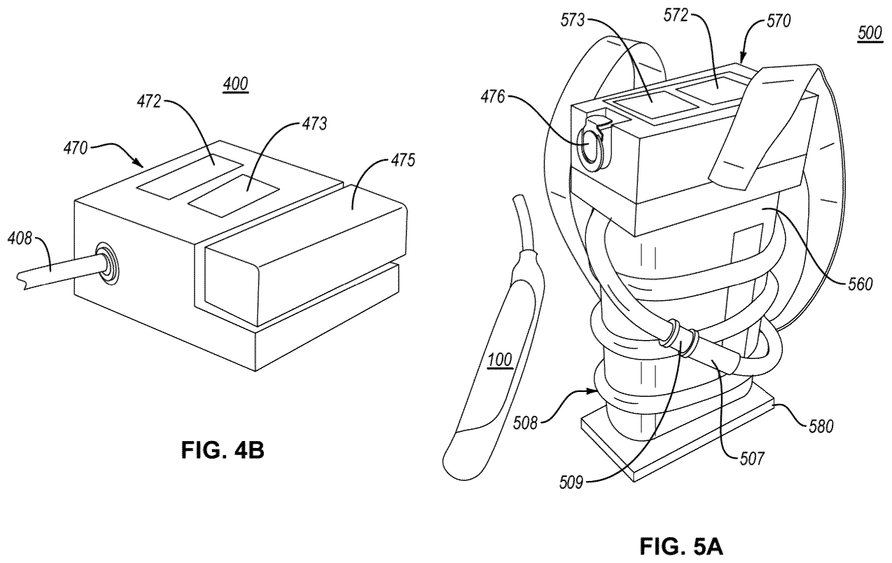

Turning now to FIGS. 4A-4B, various views of a pump unit 470 according to an embodiment are provided. FIG. 4A, for example, shows an embodiment of a fluid collection system 400 including a pump unit 470, a conduit 408 connected to the pump unit 470, the fluid collection device 100 disconnected from the conduit 408, and a fluid storage bag 460 secured to the pump unit 470. In some embodiments, the fluid collection bag 460 is selectively attachable to the pump unit 470 and reusable. For example, the fluid storage bag 460 may be attached or secured to pump unit 470, at least partially filled with fluid, detached or removed from the pump unit 470, emptied, and then reattached to the pump unit 470 (or disposed of). In some embodiments, the fluid storage bag 460 includes a valve 461 configured to allow a user to selectively empty the fluid storage bag 460 without removing the fluid storage bag 460 from the pump unit 470. The valve 460 may be generally distal to the pump unit 470.

The pump unit 470 may house any of the pumps described herein. The pump unit 470 may include a rechargeable and/or selectively removable battery. The pump unit 470 is sized, dimensioned, and weighted to be handheld by the user. In some embodiments, the pump unit 470 includes a grip on the pump unit 470 that allows a user to hold or grip the pump unit 470. The pump unit 470 may be sized, dimensioned, and otherwise configured to allow the pump unit 470 to be positioned in a personal bag (e.g., purse) or wheelchair bag of a user. Turning to FIG. 4B, the pump unit 470 includes a height of about 5.1 cm, a width of about 12.1 cm, and a depth of about 10.8 cm, according to an embodiment. In some embodiments, the pump unit 470 may include a height of about 2.5 cm to about 7.6 cm, about 2.5 cm to about 5.1 cm, about 5.1 cm to about 7.6 cm, about 2.5 cm to about 3.8 cm, about 3.8 cm to about 5.1 cm, about 5.1 cm to about 6.4 cm, about 6.4 cm to about 7.6 cm, less than about 7.6 cm, less than about 6.4 cm, less than about 5.1 cm, or less than about 3.8 cm. In some embodiments, the pump unit 470 may include a width of about 10.2 cm to about 15.2 cm, about 10.2 cm to about 12.7 cm, about 12.7 cm to about 15.2 cm, about 10.2 cm to about 11.4 cm, about 11.4 cm to about 12.7 cm, about 12.7 cm to about 14 cm, less than about 15.2 cm, less than about 14 cm, less than about 12.7 cm, or less than about 11.4 cm. In some embodiments, the pump unit 470 may include a depth of about 7.6 cm to about 12.7 cm, about 7.6 cm to about 10.2 cm, about 10.2 cm to about 12.7 cm, about 8.9 cm to about 10.2 cm, about 10.2 cm to about 11.4 cm, about 11.4 cm to about 12.7 cm, less than about 12.7 cm, less than about 11.4 cm, or less than about 10.2 cm.

Returning to FIG. 4A, the pump unit 470 may include a user interface, shown at the top of the pump unit 470 in FIG. 4A. The user interface may include an on/off button 472 that is configured to selectively activate (e.g., turn on) the pump in the pump unit 470 and selectively deactivate (e.g., turn off) the pump in the pump unit 470. The user interface also may include a battery display 473 that is configured to indicate at least one of when the battery needs to be charged, when the battery is fully charged, or a status of the battery of the pump unit. In some embodiments, battery display lights up a certain number of bars in the series of bars corresponding to the amount of charge in the battery of the pump unit 470.

The pump unit 470 may be fluidly coupled to the fluid collection device 100 with the conduit 408. The conduit 408 may include an adapter configured to secure to a conduit extending from the fluid collection device 100 effective to provide fluid communication between the chamber of the fluid collection device 100 and the conduit 408 connected to the pump unit 470. In operation, the pump unit 470 may be downstream from the fluid collection device 100, and the fluid storage bag 460 may be downstream from the pump unit 470 (e.g., the pump unit 470 is positioned between the fluid storage bag 460 and the fluid collection device 100). The pump unit 470, then, may create a negative pressure in the conduit 408 to pull fluid from the fluid collection device 100, then direct fluid into the fluid storage bag 460 attached (e.g., directly) to the pump unit 470.

In some embodiments, the conduit 408 may include a conventional conduit (e.g. tube) with the adapter configured to detachably secure to the fluid collection device 100 or a conduit extending from the fluid collection device 100. The conduit 408 and the pump unit 470 may be configured to allow the conduit 408 to be wrapped around the pump unit 470 during storage. The pump unit 470 may include an internal conduit (not visible) providing fluid communication between the (external) conduit 408 and an interior region of the fluid storage bag 460. In some embodiments, the internal conduit is a portion of the same conduit 408 that is positioned outside the pump unit 470. In some embodiments, the internal conduit is a separate conduit in fluid communication with the conduit 408 outside the pump unit 470. For example, the pump unit 470 may include a port to which the conduit 408 outside the pump unit 470 may be selectively and detachably secured. The port may provide fluid communication between the internal conduit and the conduit 408 outside the pump unit 470. In some embodiments, the pump in the pump unit 470 may be a one-way pump configured to allow fluid to pass only in the direction from the conduit 408 to the fluid storage bag 460, but prevent fluid from passing from the fluid storage bag 460 to the conduit 408. In operation, when the pump unit 470 is activated, a pump housed in the pump unit 470 may create a negative pressure in the (external) conduit 408 effective to pull fluid from the fluid collection device 100, then direct the fluid from the (external) conduit 408 through the internal conduit in the pump unit 470 and into the interior region of the fluid storage bag 460. The (external) conduit may be opaque.

The fluid storage bag 460 may be emptied using the valve 461 on the fluid storage bag 460. In some embodiments, the fluid storage bag 460 is selectively removable from the pump unit 470 for emptying. For example, in some embodiments, the pump unit 470 may include a clamp 475 configured to selectively lock or secure the fluid storage bag 460 to the pump unit 470. FIGS. 4A-4B show the clamp 475 in a locked or closed position that secures the fluid storage bag 460 to the pump unit 470 with interior of the fluid storage bag 470 in fluid communication with the pump in the pump unit 470. The clamp 470 may be pivoted to an unlocked or open position that allows the fluid storage bag 470 to be removed from the pump unit 470.

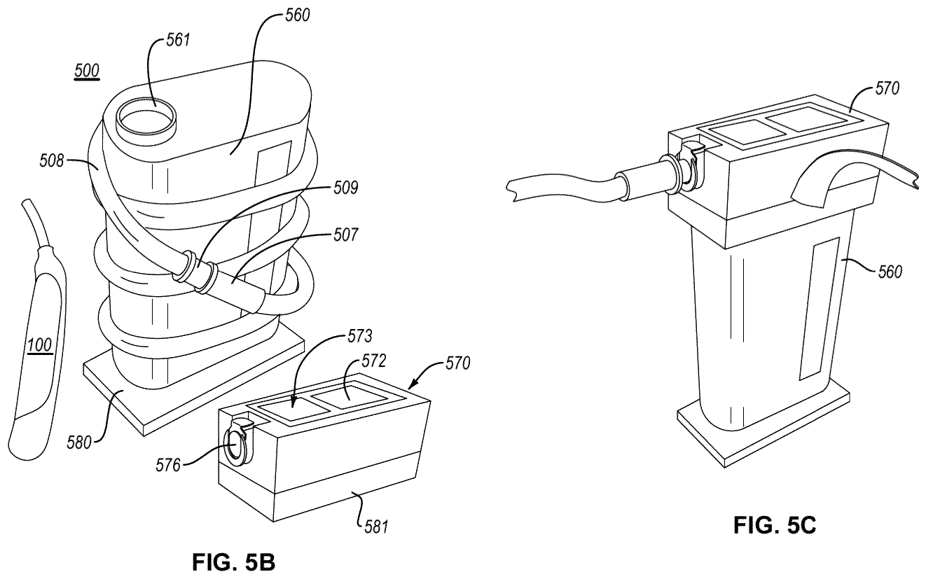

Turning now to FIGS. 5A-5C, various views of a pump unit 570 according to an embodiment are provided. FIG. 5A, for example, shows an embodiment of a fluid collection system 500 including a pump unit 570, the fluid collection device 100, a rigid fluid storage container 560 secured to the pump unit 570, and a conduit 508 disconnected from the pump unit 570 and the fluid collection device 100. In FIG. 5A, the conduit 508 wrapped around the rigid fluid storage container 560 for storage, with a first adapter 509 at a first end of the conduit 508 being detachably secured to a second adapter at a second end of the conduit 508. The first adapter 509 may be configured to secured to a conduit (not visible in FIG. 5A, but visible in FIG. 4A) extending from the fluid collection device 100 effective to provide fluid communication between the conduit of the fluid collection device 100 and the conduit 508. The pump unit 570 may include a port 576 configured to selectively receive the conduit 508 or the second adapter 507 secured to the conduit 508 effective to provide fluid communication between the pump in the pump unit 570 and the conduit 508.

In some embodiments, the pump unit 570 is detachably and selectively secured or securable to the rigid fluid storage container 560. For example, FIG. 5B shows the pump unit 570 detached from the rigid fluid storage container 560, leaving the rigid fluid storage container open for emptying. The rigid fluid storage container 560 may include a rim 561 or flange defining opening and configured to selectively and detachably secure to the pump unit 570. The rim 561 may protrude upward from a top wall of the rigid fluid storage container and may include one or more tabs. The rim 561 may be sized to fit within a port or receiver (not visible) on the pump unit 570 such that the one or more tabs on the rim 561 lock or detachably secure the rigid fluid storage container 460 to the pump unit 570. When the rigid fluid storage container 560 is secured to the pump unit 570, the opening defined by the rim 561 is positioned such that fluid pumped from the conduit 508 by the pump in the pump unit is directed through the opening defined by the rim 561 and into the interior region of the rigid fluid storage container 560. In some embodiments, the rim 561 includes a spout adapted to pour fluid from the rigid fluid storage container 560.

The pump unit 570 may house any of the pumps described herein. The pump unit 570 may include a rechargeable and/or selectively removable battery. The pump unit 570 is sized, dimensioned, and weighted to be handheld by the user. In some embodiments, the pump unit 570 includes a strap secured or securable thereto. The strap may allow the pump unit 570 to be hung from a wheelchair or carried (e.g., on the shoulder or in the hand) of the user. When secured together, the pump unit 570 and the rigid fluid storage container 560 may be shaped and configured to stand upright, such as upright on a nightstand or table. In some embodiments, a base 580 may be secured to the rigid fluid storage container 560 opposite to the pump unit 570. The base 580 may be weighted and/or otherwise shaped and configured to allow the pump unit 570 and the rigid fluid storage container 560 to stand upright on the nightstand or table.

The rigid fluid storage container 560 may be sized to fit within a cup holder with the pump unit 570 positioned above the cup holder when the rigid fluid storage container 460 is disposed within the cup holder. Turning to FIG. 5C, when the pump unit is secured to the rigid fluid storage container, the resulting assembly may together include a height of about 18.4 cm, a width of about 7.6 cm, and a depth of about 11.4 cm, according to an embodiment. In some embodiments, when the pump unit is secured to the rigid fluid storage container, the resulting assembly may together include a height of about 15.2 cm to about 20.3 cm, about 15.2 cm to about 17.8 cm, about 17.8 cm to about 20.3 cm, about 15.2 cm to about 16.5 cm, about 16.5 cm to about 17.8 cm, about 17.8 cm to about 19.1 cm, about 19.1 cm to about 20.3 cm, less than about 20.3 cm, less than about 19.1 cm, less than about 17.8 cm, or less than about 16.5 cm. In some embodiments, when the pump unit is secured to the rigid fluid storage container, the resulting assembly may together include a width of about 5.1 cm to about 10.2 cm, about 5.1 cm to about 7.6 cm, about 7.6 cm to about 10.2 cm, about 5.1 cm to about 6.4 cm, about 6.4 cm to about 7.6 cm, about 7.6 cm to about 8.9 cm, less than about 10.2 cm, less than about 8.9 cm, less than about 7.6 cm, or less than about 3.8 cm. In some embodiments, when the pump unit is secured to the rigid fluid storage container, the resulting assembly may together include a depth of about 8.9 cm to about 14 cm, about 8.9 cm to about 11.4 cm, about 10.2 cm to about 12.7 cm, about 11.4 cm to about 14 cm, about 8.9 cm to about 10.2 cm, about 10.2 cm to about 11.4 cm, about 10.8 cm to about 12.1 cm about 11.4 cm to about 12.7 cm, less than about 12.7 cm, less than about 11.4 cm, or less than about 10.2 cm.

Returning to FIGS. 5A and 5B, the pump unit may include a user interface, shown at the top of the pump unit in FIGS. 5A and 5B. The user interface may include an on/off button 572 that is configured to selectively activate (e.g., turn on) the pump in the pump unit 570 and selectively deactivate (e.g., turn off) the pump in the pump unit 570. The user interface also may include a battery display 573 that is configured to indicate the number of times the battery may be used again for individual voids. For example, the numeral 3 on the pump unit 570 of FIGS. 5A and 5B indicates the battery has enough power to pump three more voids of urine from a user.

The pump unit 570 may be fluidly coupled to the fluid collection device with the conduit 508. In FIGS. 5A and 5B, the conduit 508 is disconnected from both the pump unit 570 and the fluid collection device 100, and instead wrapped around the rigid fluid storage container 560 for storage. The pump unit 570 may include the port 576 configured to selectively and detachably secure to an outlet (e.g., the adapter 507) of the conduit 508 effective to provide fluid communication between the conduit 508 and the interior region of the rigid fluid storage container 560 and/or the pump in the pump unit 570. In some embodiments, the adapter is absent, and the conduit 508 connects directly to the port 576 on the pump unit 570.

The conduit 508 may include the adapter 509 configured to secure to a conduit extending from the fluid collection device 100 effective to provide fluid communication between the chamber of the fluid collection device 100 and the conduit 508 connected to the pump unit 570. In operation, the pump unit 570 may be downstream from the fluid collection device 100, and the rigid fluid storage container 560 may be downstream from the pump unit 570 (e.g., the pump unit is positioned between the rigid fluid storage container 560 and the fluid collection device 100). The pump unit 570, then, may create a negative pressure in the conduit 508 to pull fluid from the fluid collection device 100, then direct fluid into the interior region of the rigid fluid storage container 560 attached (e.g., directly) to the pump unit 570.

In some embodiments, the conduit 508 may include a conventional conduit (e.g. tube) with the adapter 509 configured to detachably secure to the fluid collection device 100 or a conduit extending from the fluid collection device 100. The pump unit 570 may include an internal conduit providing fluid communication between the (external) conduit 508 and an interior region of the rigid fluid storage container 560. In some embodiments, the internal conduit is a portion of the same conduit 508 that is positioned outside the pump unit 570. In some embodiments, the internal conduit is a separate conduit in fluid communication with the conduit 508 outside the pump unit 570. For example, the pump unit 570 may include the port 576 to which the conduit 508 outside the pump unit 570 may be selectively and detachably secured. The port 576 may provide fluid communication between the internal conduit and the conduit 508 outside the pump unit 570. In operation, when the pump unit 570 is activated, a pump housed in the pump unit 570 may create a negative pressure in the (external) conduit 508 effective to pull fluid from the fluid collection device 100, then direct the fluid from the (external) conduit 508 through the internal conduit in the pump unit 570 and into the interior region of the rigid fluid storage container 560. In some embodiment, the pump in the pump unit 570 is configured to allow only one-way movement of fluid from the conduit 508 to the rigid fluid storage container 560, and prevent movement of fluid from the rigid fluid storage container 560 to the conduit 508.

The rigid fluid storage container 560 may be emptied by detaching the rigid fluid storage container 560 from the pump unit 570. In some embodiments, the rigid fluid storage container includes a window that allows a user to see the amount of fluid in the rigid fluid storage container 560 (e.g., determine if the rigid fluid storage container is full and/or needs to be emptied). At least one of the rigid fluid storage container 560 or the pump unit 570may include one or more lips or flanges (e.g., the protruding rim 561) configured to lock the rigid fluid storage container 560 to the pump unit 570. The (external) conduit 508 may be tinted or opaque.

As used herein, the term “about” or “substantially” refers to an allowable variance of the term modified by “about” by +10% or +5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection system, comprising:
a fluid collection device having a chamber and configured to collect fluid from a user in the chamber;
a first conduit in fluid communication with the chamber of the fluid collection device;
a pump unit connected or connectable to the first conduit and including a pump configured to create a negative pressure to draw fluid from the chamber of the fluid collection device through the first conduit, wherein the pump unit is sized, dimensioned, and weighted to be handheld by the user; and
a fluid storage container having an interior region in fluid communication with the first conduit and configured to collect fluid drawn from the chamber of the fluid collection device through the first conduit.

2. The fluid collection system of claim 1, further comprising:
wherein the fluid storage container includes a fluid storage bag having the interior region;
a second conduit extending between the pump unit and the fluid storage bag; and
an adapter plug secured or securable to the pump unit and having the first conduit and the second conduit extending therefrom.

3. The fluid collection system of claim 2, wherein:
the pump unit includes a recess shaped complementary to the adapter plug and configured to receive the adapter plug;
the adapter plug includes a first prong in fluid communication with the first conduit and a second prong in fluid communication with the second conduit;
the recess includes a first port positioned to receive the first prong when the adapter plug is positioned within the recess effective to provide fluid communication between the pump and the first conduit; and
the recess includes a second portion positioned to receive the second prong when the adapter plug is positioned within the recess effective provide fluid communication between the pump and the second conduit.

4. The fluid collection system of claim 2, wherein the pump unit includes a width of about 6.5 cm to about 8.5 cm, a depth of about 5 cm to about 6.5 cm, and a height of about 12 cm to about 14 cm.

5. The fluid collection device of claim 4, wherein the pump unit is elongated and includes a bottom region configured to rest on a flat surface and an upper region opposite to the bottom region, the upper region including the recess configured to receive the adapter plug such that the when the adapter plug is disposed in the recess the first conduit and the second conduit extend upward from the upper region of the pump unit.

6. The fluid collection system of claim 2, wherein the pump unit is sized for storage during use in a cup holder or secured to an arm rest of a chair or wheelchair with a handle on the pump unit.

7. The fluid collection system of claim 1, wherein the fluid storage container includes a fluid storage bag secured or securable to the pump unit such that the pump in the pump unit is positioned to direct fluid from the conduit into the fluid storage bag.

8. The fluid collection system of claim 7, wherein the fluid storage bag is secured or securable to the pump unit such that the fluid collection system of devoid of an additional conduit extending from the pump unit to the fluid storage bag when the fluid storage bag is secured to the pump unit.

9. The fluid collection system of claim 7, wherein the pump unit includes a clamp selectively pivotable or adjustable between a closed position securing the fluid storage bag to the pump unit and an open position that allows the fluid storage bag to be removed or disconnected from the pump unit.

10. The fluid collection system of claim 7, wherein the fluid storage bag is reusable and includes a valve distal to the pump unit when the fluid storage bag is secured to the pump unit.

11. The fluid collection system of claim 7, wherein the pump unit includes a depth of about 10 cm to about 12 cm, a width of about 11 cm to about 13 cm, and a height of about 4 cm to about 6 cm.

12. The fluid collection system of claim 7, wherein the pump unit is sized for storage during use in a personal handbag or a wheelchair bag.

13. The fluid collection system of claim 1, wherein fluid storage container includes a rigid fluid storage container detachably secured or securable to the pump unit.

14. The fluid collection system of claim 13, wherein the rigid fluid storage container is elongated.

15. The fluid collection system of claim 13, wherein:
the rigid fluid storage container includes an upper wall having a rim or flange protruding from the upper wall, the rim or flange defining an opening in the rigid fluid storage container in fluid communication with the interior region of the rigid fluid storage container; and
the pump unit is configured to receive the rim or flange when the rigid fluid storage container is secured to the pump unit such that the opening is positioned to receive fluid pumped from the conduit by the pump.

16. The fluid collection system of claim 15, wherein the rim or flange includes one or more tabs configured to lock or secure the rigid fluid storage container to the pump unit.

17. The fluid collection system of claim 15, wherein the rim or flange includes a pouring spout.

18. The fluid collection system of claim 13, wherein the pump unit includes a port configured to selectively secure the conduit to the pump unit and provide fluid communication between the conduit and the pump in the pump unit.

19. The fluid collection system of claim 13, wherein when the pump unit is secured to the rigid fluid storage container, the pump unit and the rigid fluid storage container together include a width of about 10.5 cm to about 12.5 cm, a depth of about 6.5 cm to about 8.5 cm, and height of about 17.5 cm to about 19.5 cm.

20. The fluid collection system of claim 13, wherein when the pump unit is secured to the rigid fluid storage container, the pump unit and the rigid fluid storage container together are sized for storage during use in a personal handbag or a wheelchair bag.

21. The fluid collection system of claim 1, wherein the pump unit includes a handle sized and dimensioned to hang from an armrest on a wheelchair.

22. A fluid collection system, comprising:
a fluid collection device having a chamber and configured to collect fluid from a user in the chamber;
a first conduit in fluid communication with the chamber of the fluid collection device;
a handheld pump unit connected or connectable to the first conduit and including a pump configured to create a negative pressure to draw fluid from the chamber of the fluid collection device through the first conduit; and
a fluid storage bag having an interior region in fluid communication with the first conduit and configured to collect fluid drawn from the chamber of the fluid collection device through the first conduit,
wherein the handheld pump includes a depth of about 5 cm to about 12 cm, a width of about 6.5 cm to about 13 cm, and a height of about 4 cm to about 14 cm such that the handheld pump unit is sized, dimensioned, and weighted to be handheld by the user.

23. The fluid collection system of claim 22, wherein the pump unit includes a width of about 6.5 cm to about 8.5 cm, a depth of about 5 cm to about 6.5 cm, and a height of about 12 cm to about 14 cm.

24. The fluid collection system of claim 22, further comprising:
a second conduit extending between the pump unit and the fluid storage bag;
a recess in an upper region of the pump unit, the recess including a first port and a second port;
an adapter plug shaped complementary to the recess in the upper region of the pump unit and secured or securable to the pump unit, the adapter plug having the first conduit and the second conduit extending therefrom, a first prong in fluid communication with the first conduit and configured to be disposed within the first port in the recess effective to provide fluid communication between the pump and the first conduit, and a second prong in fluid communication with the second conduit and configured to be disposed within the second port in the recess effective provide fluid communication between the pump and the second conduit.

25. The fluid collection system of claim 22, wherein the pump unit includes a depth of about 10 cm to about 12 cm, a width of about 11 cm to about 13 cm, and a height of about 4 cm to about 6 cm.

26. The fluid collection system of claim 25, wherein fluid storage bag secured or securable to the pump unit such that the pump in the pump unit is positioned to direct fluid from the conduit into the fluid storage bag and the fluid collection system of devoid of an additional conduit extending from the pump unit to the fluid storage bag when the fluid storage bag is secured to the pump unit, the fluid storage bag being reusable and having a valve distal to the pump unit when the fluid storage bag is secured to the pump unit.

27. The fluid collection system of claim 25, wherein the pump unit includes a clamp selectively pivotable or adjustable between a closed position securing the fluid storage bag to the pump unit and an open position that allows the fluid storage bag to be removed or disconnected from the pump unit.

28. A fluid collection system, comprising:
a fluid collection device having a chamber and configured to collect fluid from a user in the chamber;
a conduit in fluid communication with the chamber of the fluid collection device;
a handheld pump unit connected or connectable to the conduit and including a pump configured to create a negative pressure to draw fluid from the chamber of the fluid collection device through the conduit; and
a rigid fluid storage container secured or securable to the pump unit and having an interior region in fluid communication with the conduit and configured to collect fluid drawn from the chamber of the fluid collection device through the conduit,
wherein when the pump unit is secured to the rigid fluid storage container, the pump unit and the rigid fluid storage container together include a width of about 10.5 cm to about 12.5 cm, a depth of about 6.5 cm to about 8.5 cm, and height of about 17.5 cm to about 19.5 cm such that the handheld pump unit is sized, dimensioned, and weighted to be handheld by the user.

29. The fluid collection system of claim 28, wherein:
the rigid fluid storage container includes an upper wall having a rim or flange protruding from the upper wall, the rim or flange defining an opening in the rigid fluid storage container in fluid communication with the interior region of the rigid fluid storage container and including one or more tabs configured to lock or secure the rigid fluid storage container to the pump unit; and
the pump unit is configured to receive the rim or flange when the rigid fluid storage container is secured to the pump unit such that the opening is positioned to receive fluid pumped from the conduit by the pump.

30. The fluid collection system of claim 29, wherein the rim or flange includes a pouring spout.

31. The fluid collection system of claim 28, wherein the pump unit includes a port configured to selectively secure the conduit to the pump unit and provide fluid communication between the conduit and the pump in the pump unit.

32. (canceled)
